(19) United States
(12) Patent Application Publication
(10) Pub. No.: US 2026/0248650 A1
(43) Pub. Date: Aug. 27, 2026

(54) HYBRID LASER DELIVERY

(71) Applicant: Alcon Inc., Fribourg (CH)

(72) Inventors: Alireza Mirsepassi, Irvine, CA (US); Qing Xiang, Irvine, CA (US)

(21) Appl. No.: 19/460,534

(22) Filed: Jan. 27, 2026

Related U.S. Application Data
(60) Provisional application No. 63/763,365, filed on Feb. 26, 2025.

(51) Int. Cl. A61F 9/008

(52) U.S. Cl. CPC A61F 9/008; A61F 2009/00863; A61F 2009/00874

(57) ABSTRACT

A laser system for use in various surgical procedures. A system may include a laser source, an arm having a free-space optical path coupled to the laser source, and a cable having a waveguide coupled to the free-space optical path.

### TECHNICAL FIELD

The invention set forth in the appended claims relates generally to laser delivery, including, without limitation, apparatus, systems, and methods for delivering infrared laser for surgical procedures.

### BACKGROUND

Lasers are used in many different medical procedures, including various ophthalmic procedures. For example, lasers may be used in cataract surgery, such as for fragmenting the cataractous lens. In some procedures, a laser may be used for fragmentation of the lens, followed by phacoemulsification of the lens by an ultrasonic handpiece to complete the breakdown of the lens for removal. In other procedures, a laser may be used for complete fragmentation or phacoemulsification of the lens for removal, without the need for a separate application of ultrasonic energy. A laser may also be used in vitreoretinal surgery. In some procedures, a laser may be used for vitrectomy, to sever or break the vitreous fibers for removal.

While the benefits of using a laser in surgical procedures are known, improvements to laser systems, components, and processes can continue to improve outcomes and benefit patients.

### BRIEF SUMMARY

New and useful systems, apparatuses, and methods for laser delivery are set forth in the appended claims. Illustrative embodiments are also provided to enable a person skilled in the art to make and use the claimed subject matter.

For example, some embodiments are related to laser energy delivery from a light source to a patient eye in an ophthalmic surgical instrument. Some embodiments may comprise a hybrid laser energy delivery method, which may include a free-space delivery and a short fiber delivery. In more specific embodiments, the free-space delivery may be an articulating arm with a fiber optical coupling interface, which can deliver laser energy from a laser source to an operation table near the eye. A short optical fiber can deliver the laser energy from the operation table into the eye. The arm may be flexible so that it can be positioned anywhere near the eye, and the fiber length may be less than half a meter in some examples. The short fiber may be a straight or a tapered fiber. A taper can help mode size adaption, reduce mode mismatch, and improve alignment loss. In some examples, the optical fiber coupling interface may include a fiber auto-alignment system, which can check the back reflection from the fiber to provide high-efficiency auto-alignment.

In some examples, the arm may be articulating, foldable, retractable, or some combination thereof. In some examples, the arm may be combined with a surgical microscope arm or placed at the front end of the surgical microscope arm. For example, the optical fiber interface may be located aside the objective lens of the microscope.

More generally, some embodiments may relate to a system for surgery on an eye, and the system may include a laser source, an arm having a free-space optical path coupled to the laser source, and a cable having a waveguide coupled to the free-space optical path. In more particular embodiments, the laser source may be configured to deliver a laser to the free-space optical path, and the laser may have a wavelength in a range of about 2.5 microns to about 3.5 microns. The cable may have a length that is less than 2 meters and may be less than or equal to 0.5 meters in some embodiments. In some embodiments, the cable may be an optic fiber comprising or consisting essentially of sapphire or germanite. The arm may include at least one joint and at least one reflector configured to move with the joint to maintain alignment of the laser along the free-space optical path. In some embodiments, the joint may be an articulating joint, a folding joint, a telescoping joint, or some combination thereof. The cable may be tapered in some embodiments.

Some embodiments may relate to a system comprising a laser source configured to generate a laser having a wavelength in a range of about 2.5 microns to about 3.5 microns, a first optical conductor having a free-space optical path coupled to the laser source, and a second optical conductor having a waveguide coupled to the free-space optical path. The second optical conductor may include sapphire or germanite and have a length that is less than 2 meters. The first optical conductor may include at least one joint and at least one reflector configured to move with the joint to maintain alignment of the laser along the free-space optical path. Additionally, or alternatively, the joint may be an articulating joint, a folding joint, a telescoping joint, or some combination thereof. The waveguide may be tapered in some embodiments.

A method for delivering a laser to a surgical target may include: providing a laser source configured to generate a laser having a wavelength in a range of about 2.5 microns to about 3.5 microns; providing a first optical conductor having a free-space optical path coupled to the laser source; providing a second optical conductor having a waveguide coupled to the free-space optical path; and transmitting the laser from the laser source through the free-space optical path and the waveguide to the surgical target. The second optical conductor may include sapphire or germanite and have a length that is less than 2 meters.

Features, elements, and aspects described in the context of some embodiments may also be omitted, combined, or replaced by alternative features. Other features, objectives, advantages, and modes of making and using the claimed subject matter are described in greater detail below with reference to the accompanying drawings of illustrative embodiments.

### BRIEF DESCRIPTION OF THE DRAWINGS

The accompanying drawings illustrate some objectives, advantages, and modes of making and using some embodiments of the claimed subject matter. Like reference numbers represent like parts in the examples.

FIG. 1 is a schematic diagram of an example of a laser system for use in various surgical procedures.

FIG. 2 is a schematic diagram of another example of a laser system for use in various surgical procedures.

FIG. 3 is a schematic diagram of a method for using an example embodiment of the laser system of FIG. 1 to treat a surgical target.

### DESCRIPTION OF EXAMPLE EMBODIMENTS

The following description of example embodiments provides information that enables a person skilled in the art to make and use the subject matter set forth in the appended claims. The following detailed description is, therefore, to be taken as illustrative and not limiting.

The example embodiments may also be described herein with reference to spatial relationships between various elements or to the spatial orientation of various elements depicted in the attached drawings. In general, such relationships or orientation assume a frame of reference consistent with or relative to a patient in a position for ophthalmic surgery. However, as should be recognized by those skilled in the art, this frame of reference is merely a descriptive expedient rather than a strict requirement.

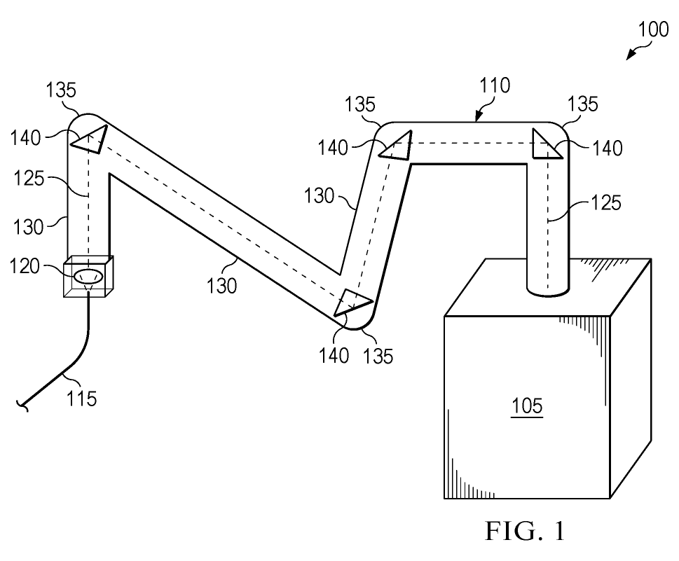

FIG. 1 is a schematic diagram of an example of a laser system 100 for use in various surgical procedures. As shown in the example of FIG. 1, some embodiments of the laser system 100 may comprise a laser source 105, an arm 110, and a cable 115. The arm 110 may be coupled to the laser source 105, and the cable 115 may be coupled to the arm at the opposite end. The laser system 100 of FIG. 1 also includes a coupling 120, which may be disposed between the arm 110 and the cable 115.

The laser source 105 may provide any type of electromagnetic radiation at any type of wavelength suitable for the desired application. For example, the laser source 105 may emit electromagnetic radiation in one or more wavelengths in the visible, infrared, and/or ultraviolet wavelengths, referred to herein as a “laser”. The laser source 105 may operate or be operated to emit a continuous beam of electromagnetic radiation, a pulsed beam, or a combination of both.

In some examples, the laser source 105 may provide electromagnetic radiation in the infrared range. For example, the laser source 105 may output electromagnetic radiation in the mid-infrared range. A range of about 2.0 microns to about 4.0 microns may be used for some types of treatment. Some example wavelengths include about 2.5 microns to 3.5 microns, such as about 2.775 microns, about 2.8 microns, or about 3.0 microns. In other examples, the laser source 105 may emit electromagnetic radiation in the ultraviolet range or the visible range. Such a laser may be suitable, for example, for lens fragmentation in cataract surgery, or for other procedures.

Some components of the laser system 100 may be housed within or used in conjunction with other components, such as sensors, imaging units, processing units, alarm indicators, memory, databases, software, display devices, or user interfaces that further facilitate use of the laser system 100. For example, in some embodiments, the laser source 105 may be combined with a controller, sensors, and other components into a console or other housing.

The laser source 105 of FIG. 1 may be configured to direct a laser 125 to one or more optical conductors. An “optical conductor,” in this context, broadly includes any structure adapted to convey light between two ends. Some optical conductors may be molded into or otherwise integrally combined with other components. Some optical conductors may provide a free-space optical path (i.e., open air) between two ends. For example, a free-space conductor may be a tube, pipe, hose, conduit, or other structure with one or more lumina or open pathways adapted to convey light between two ends. Typically, a free-space conductor may be an elongated, cylindrical structure with some flexibility, but the geometry and rigidity may vary. Some optical conductors may provide a waveguide that can convey light between two ends. For example, an optic fiber may provide a waveguide. Typically, an optic fiber may comprise or consist essentially of a core surrounded by a cladding. The refractive index of the core must be greater than that of the cladding to confine a laser in the core. In some examples, the core may be a monofilament of flexible material, such as glass or plastic. For visible and near infrared wavelength laser, silica glass may be a suitable material for the core. For a middle infrared (Mid-IR) laser, other materials may be advantageous. For example, chalcogenide glass, germanite glass, heavy metal fluoride glass, polycrystalline fiber, sapphire fiber, etc., may be used.

The arm 110 can provide a free-space optical path with a controlled environment. For example, the free space of the optical path may consist of air, which can be controlled for pressure, humidity, and other parameters for safety, cleanliness, and efficiency. In some examples, the free space optical path may be defined, at least in part, by a conduit within or hollow interior of the arm 110. The arm 110 may have at least two segments 130, which can be coupled by joints 135. The joints 135 can allow the position and configuration of the arm 110 to be adjusted for many applications. For example, at least some of the joints 135 may be articulating joints. In some examples, the arm 110 may be motorized and manipulated by a controller. One or more reflectors 140, such as a mirror or prism, may be positioned in the arm 110 to reflect the laser 125 along the optical path. The reflectors 140 may be configured to move with the joints 135 to maintain alignment of the laser 125 along the optical path if the joints 135 are moved. In some embodiments, the arm 110 can provide an optical path with a fixed distance between the laser source 105 and the coupling 120, which can provide the laser 125 with a fixed width at the coupling 120. The arm 110 may be sealed to provide an environmentally controlled environment for the optical path.

The cable 115 may provide a waveguide that can transmit a laser along the axis of the cable 115. For example, the cable 115 may be an optical fiber. The cable 115 of FIG. 1 may be straight or tapered. In some embodiments, a taper may help mode size adaption, reduce mode mismatch, and reduce alignment loss. The length of the cable 115 may vary according to application. A length of less than two (2) meters may be used for some examples. In some examples, the length may be less than one-half (0.5) meter (other lengths are also contemplated).

The coupling 120 can be configured to transfer the laser 125 from the arm 110 to the cable 115. For example, the coupling 120 may have one or more lenses configured to focus the laser 125 into the cable 115. In some examples, the coupling 120 may include an alignment system, which can check the back reflection from the cable 115 to improve alignment efficiency.

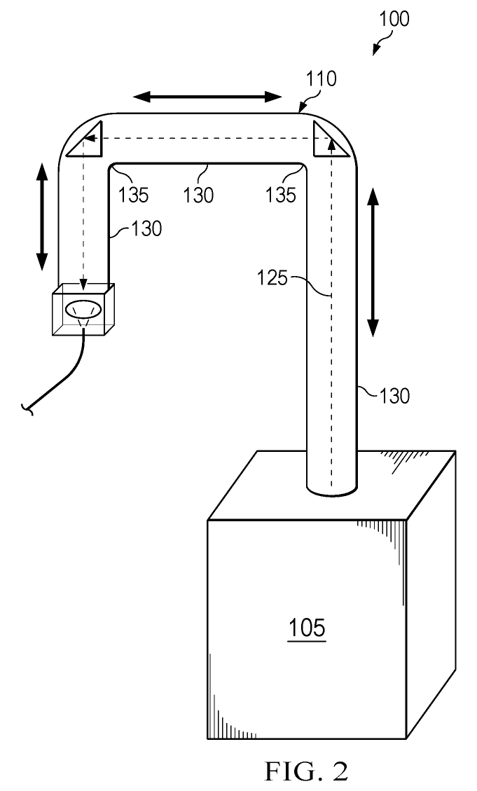

FIG. 2 is a schematic diagram of another example of the laser system 100. In the example of FIG. 2, at least some of the joints 135 may be telescoping joints that can be extended and retracted to adjust the length of one or more of the segments 130 of the arm 110.

In general, components of the laser system 100 may be coupled directly or indirectly. For example, the laser source 105 may be directly coupled to the arm 110 and may be indirectly coupled to the cable 115 through the arm 110. Coupling may include optical, mechanical, thermal, electrical, or chemical coupling (such as a chemical bond), or some combination of coupling in some contexts. For example, the laser source 105 may be optically and mechanically coupled to the arm 110. In some embodiments, components may also be coupled by virtue of physical proximity, being integral to a single structure, or being formed from the same piece of material.

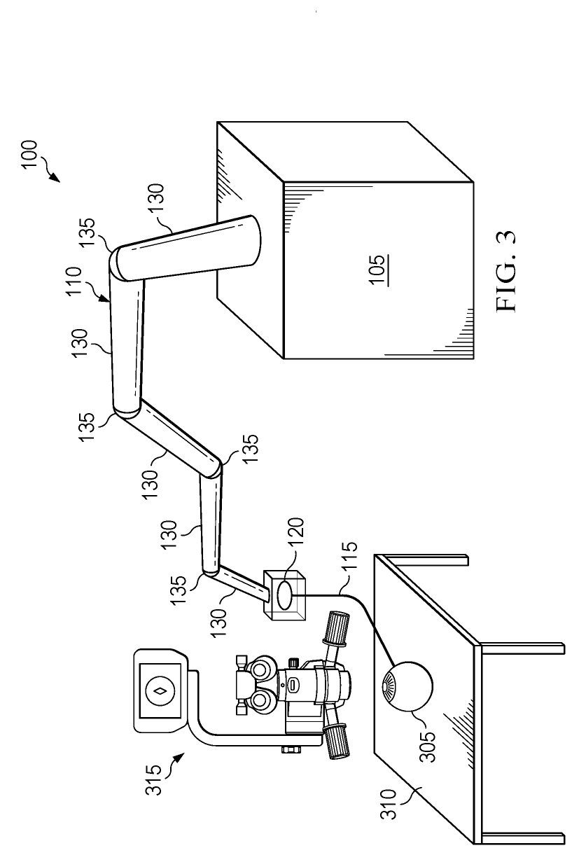

FIG. 3 is a schematic diagram of a method for using an example embodiment of the laser system 100 to treat a surgical target, such as an eye 305. In operation, a patient may be positioned on his back on a bed or other surgical platform 310. A surgical microscope 315 may be aligned with the eye 305. In the example of FIG. 3, the arm 110 is an articulating arm, and the joints 135 may be adjusted to position the coupling 120 near the eye 305. In some examples, it may be advantageous to attach the coupling 120 or a distal portion of the arm 110 to the surgical microscope 315. The laser source 105 may be operated to generate the laser 125, which can be directed through the arm 110 to the cable 115. In some examples, the arm 110 may be purged with nitrogen or other suitable gas to reduce humidity in the arm 110 before operation. The cable 115 may deliver a laser from the arm 110 to the eye 305. In some examples, a patient interface, such as a handpiece, may be coupled to the cable 115 to facilitate control of the laser 125 output from the cable 115.

The systems, apparatuses, and methods described herein may provide significant advantages. For example, silica fiber is well developed with high transmittance and low cost and may be advantageous for a laser in the visible and near-infrared wavelengths, but it may not be well suited for a laser having a wavelength in the Mid-IR range, such as a 3-micron laser. Other fibers can be used for Mid-IR lasers, such as chalcogenide glass, germanite glass, heavy metal fluoride glass, polycrystalline fiber, and sapphire fiber. However, the loss of these optical fibers is still high compared to silica glass fiber in the visible wavelength range. For example, a loss of about 50%-60% may be expected for a 3-micron laser with sapphire fiber of about two meters. A sapphire fiber also does not have a cladding and can make transmission less consistent and more sensitive to moisture and contamination. A sapphire fiber is also more expensive than silica. Since fiber may be used (e.g., only once) to eliminate potential for cross-contamination and infection, the expense of sapphire fiber can make it impractical for a single use. The losses in germanite glass fiber may be less than sapphire, but it may be moisture sensitive, brittle, and can be difficult to handle. The design and manufacture of germanite fiber can be complicated and expensive, which can also make a single use of these optical cables impractical for many applications. Multiple uses would require autoclaving.

The laser system 100 can significantly reduce the length of the fiber needed to deliver a laser to a surgical target, which may be particularly advantageous for a Mid-IR laser. Reducing the length of the fiber can significantly reduce the cost and transmission losses for Mid-IR fiber, such as sapphire fiber and germanite. Additionally, or alternatively, the arm 110 and the coupling 120 may be non-consumable equipment, while the length of the cable 115 can make single use more practical. Single use of the cable 115 can eliminate the need for an autoclave process.

While shown in a few illustrative embodiments, a person having ordinary skill in the art will recognize that the systems, apparatuses, and methods described herein are susceptible to various changes and modifications that fall within the scope of the appended claims.

Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use.

The claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

What is claimed is:

1. A system for surgery on an eye, the system comprising:
a laser source;
an arm having a free-space optical path coupled to the laser source; and
a cable having a waveguide coupled to the free-space optical path.

2. The system of claim 1, wherein:
the laser source is configured to deliver a laser to the free-space optical path; and
the laser has a wavelength in a range of about 2.5 microns to about 3.5 microns.

3. The system of claim 1, wherein the cable has a length that is less than 2 meters.

4. The system of claim 1, wherein:
the cable is an optic fiber comprising sapphire or germanite; and
the cable has a length that is less than 2 meters.

5. The system of claim 4, wherein the length is less than or equal to 0.5 meters.

6. The system of claim 1, wherein:
the laser source is configured to deliver a laser to the free-space optical path; and
the arm comprises at least one joint and at least one reflector configured to move with the joint to maintain alignment of the laser along the free-space optical path.

7. The system of claim 6, wherein the joint is an articulating joint.

8. The system of claim 6, wherein the joint is a telescoping joint.

9. The system of claim 1, wherein the cable is tapered.

10. A system for surgery on an eye, the system comprising:
a laser source configured to generate a laser having a wavelength in a range of about 2.5 microns to about 3.5 microns;
a first optical conductor having a free-space optical path coupled to the laser source; and
a second optical conductor having a waveguide coupled to the free-space optical path;
wherein second optical conductor comprises sapphire or germanite and has a length that is less than 2 meters.

11. The system of claim 10, wherein the first optical conductor comprises at least one joint and at least one reflector configured to move with the joint to maintain alignment of the laser along the free-space optical path.

12. The system of claim 11, wherein the joint is an articulating joint.

13. The system of claim 11, wherein the joint is a telescoping joint.

14. The system of claim 11, wherein the waveguide is tapered.

15. A method for delivering a laser to a surgical target; the method comprising:
providing a laser source configured to generate a laser having a wavelength in a range of about 2.5 microns to about 3.5 microns;
providing a first optical conductor having a free-space optical path coupled to the laser source;
providing a second optical conductor having a waveguide coupled to the free-space optical path, wherein the second optical conductor comprises sapphire or germanite and has a length that is less than 2 meters; and
transmitting the laser from the laser source through the free-space optical path and the waveguide to the surgical target.
